(19) United States
(12) Patent Application Publication
(10) Pub. No.: US 2024/0099936 A1
(43) Pub. Date: Mar. 28, 2024

(54) LIQUID TRANSFER DEVICE WITH INTEGRATED SYRINGE

(71) Applicant: WEST PHARMA. SERVICES IL, LTD., Ra'anana (IL)

(72) Inventor: Igor DENENBURG, Gedera (IL)

(21) Appl. No.: 17/767,906

(22) PCT Filed: Mar. 26, 2020
(86) PCT No.: PCT/IL2020/050362

§ 371 Date: Apr. 10, 2022

Related U.S. Application Data
(60) Provisional application No. 62/831,214, filed on Apr. 9, 2019.

(51) Int. Cl. A61J 1/10; A61J 1/20

(52) U.S. Cl. CPC A61J 1/10; A61J 1/2013; A61J 1/2055; A61J 1/2062; A61J 1/2096

(57) ABSTRACT

A liquid transfer device is provided for mixing a medicament additive contained within a vial with an infusion liquid contained within an infusion liquid container to form a medicated infusion liquid. The liquid transfer device includes a trifurcated connector body defining a barrel at a first end thereof, an IV spike at a second end thereof and a vial adapter lumen with a vial adapter secured thereto at a third end thereof. A plunger having an IV port is slidably engaged with the barrel to form a syringe integrated with the device. The syringe enables the device to flush out the flow path between the IV port and the IV spike connected to the medicated infusion liquid, and any residual unmixed medicament additive therein, after combining the medicament additive with the infusion liquid.

### CROSS-REFERENCE TO RELATED APPLICATIONS

This application claims priority from U.S. Provisional Patent Application No. 62/831,214, titled “Liquid Transfer Device With Integrated Syringe”, filed on Apr. 9, 2019, the entire contents of which are incorporated by reference herein.

### BACKGROUND OF THE DISCLOSURE

The present disclosure is generally directed to liquid transfer devices.

Conventional infusion liquid containers containing an infusion liquid to be delivered to a patient generally take the form of an infusion liquid bag, an infusion liquid bottle, and the like. A pre-filled syringe or vial is generally utilized to add a high concentration of a drug to the infusion liquid contents, via a liquid transfer device, to form a diluted, medicated infusion liquid. Thereafter, an infusion set including an IV spike is generally inserted into an IV port of the liquid transfer device for infusion of medicated infusion liquid contents to a patient.

It would be advantageous to manufacture a liquid transfer device capable of flushing the flow path between the IV port and the port connected to the medicated infusion liquid contents between the step of adding/mixing the liquid drug to the infusion liquid contents and the step of administering the medicated infusion liquid contents to a patient, as an added measure to prevent the patient from receiving a portion of the drug in an undiluted, high concentration form.

### BRIEF SUMMARY OF THE DISCLOSURE

Briefly stated, one aspect of the present disclosure is directed to a liquid transfer device configured for use with each of an infusion liquid container having an intravenous (IV) port, a vial sealed by a vial stopper, and an infusion set including an IV spike for sealing insertion into an IV port. The liquid transfer device includes a trifurcated connector body defining a barrel at a first end thereof, an IV spike at a second end thereof and a vial adapter lumen at a third end thereof. The barrel defines an internal chamber having an open proximal end. The IV spike defines an internal IV spike lumen having a distally located first aperture, and the IV spike is configured to sealingly insert into the IV port of the infusion liquid container. The vial adapter lumen branches off of the IV spike lumen. A vial adapter is secured to the vial adapter lumen and configured to mount onto the vial. The vial adapter includes a cannula fluidly connected with the vial adapter lumen and configured to puncture the vial stopper upon mounting of the vial adapter onto the vial for flow communication therewith. A plunger is in slidable engagement with the internal chamber of barrel via the open proximal end thereof. The plunger includes a piston face transversely oriented relative to, and in sealing engagement with, the barrel and a tube projecting proximally from the piston face and terminating in an IV port configured to sealingly receive the IV spike of the infusion set. The tube defines an internal plunger lumen therein. A plunger rod projects distally from the piston face and into the IV spike lumen. The plunger rod and the IV spike lumen define a fluid-flow pathway therebetween, the fluid-flow pathway being sealed off from the vial adapter lumen. A second aperture is interposed between a distal end of the internal chamber of the barrel and a proximal end of the IV spike lumen, and aligned with the fluid-flow pathway, to fluidly connect the fluid-flow pathway with the internal chamber of the barrel. In an advanced position of the plunger, the plunger rod seals off the vial adapter lumen from fluid communication with the IV spike lumen and the internal chamber of the barrel is in fluid communication with the IV spike lumen and the first aperture of the IV spike via the fluid-flow pathway and the second aperture. In a retracted position of the plunger, the plunger rod seals off the internal chamber of the barrel from fluid communication with the IV spike lumen and the first aperture of the IV spike, and the vial adapter lumen is fluidly communicated with the IV spike lumen and the first aperture of the IV spike.

Briefly stated, another aspect of the present disclosure is directed to a method of mixing a medicament additive contained within a vial and sealed by a vial stopper with an infusion liquid contained within an infusion liquid container, with a liquid transfer device, to form a medicated infusion liquid. The liquid transfer device includes a trifurcated connector body defining a barrel at a first end thereof, an IV spike at a second end thereof and a vial adapter lumen at a third end thereof. the barrel defines an internal chamber having an open proximal end. The IV spike defines an internal IV spike lumen having a distally located first aperture, and the vial adapter lumen branches off of the IV spike lumen. A vial adapter is secured to the vial adapter lumen, and includes a cannula fluidly connected with the vial adapter lumen. A plunger is in slidable engagement with the internal chamber of the barrel via the open proximal end thereof. The plunger includes a piston face transversely oriented relative to, and in sealing engagement with the barrel, and a tube projecting proximally from the piston face and terminating in an IV port. The tube defines an internal plunger lumen therein. A plunger rod projects distally from the piston face and into the IV spike lumen. The plunger rod and the IV spike lumen define a fluid-flow pathway therebetween, the fluid-flow pathway being sealed off from the vial adapter lumen. A second aperture is interposed between a distal end of the internal chamber of the barrel and a proximal end of the IV spike lumen, and aligned with the fluid-flow pathway to fluidly connect the fluid-flow pathway with the internal chamber of the barrel. In an advanced position of the plunger, the plunger rod seals off the vial adapter lumen from fluid communication with the IV spike lumen and the internal chamber of the barrel is in fluid communication with the IV spike lumen and the first aperture of the IV spike via the fluid-flow pathway and the second aperture. In a retracted position of the plunger, the plunger rod seals off the internal chamber of the barrel from fluid communication with the IV spike lumen and the first aperture of the IV spike, and the vial adapter lumen is fluidly communicated with the IV spike lumen and the first aperture of the IV spike. The method includes the steps of:
(i) sealingly inserting the IV spike of the liquid transfer device into an IV port of the infusion liquid container;
(ii) mounting the vial adapter onto the vial and puncturing the vial stopper for flow communication between the vial and the vial adapter lumen;
(iii) withdrawing the plunger proximally through the internal chamber of the barrel, from the advanced position thereof toward the retracted position thereof, and, in turn, pulling a volume of the infusion liquid into the internal chamber of the barrel;
(iv) sealing off the internal chamber of the barrel from fluid communication with the IV spike lumen and the first aperture of the IV spike upon reaching the retracted position of the plunger;
(v) combining the medicament additive contained within the vial with the infusion liquid within the infusion liquid container via fluid communication between the vial adapter lumen, the IV spike lumen and the first aperture of the IV spike when the plunger is in the retracted position, to form a medicated infusion liquid; and
(vi) depressing the plunger distally through the internal chamber to re-seal the vial adapter lumen from fluid communication with the IV spike lumen, re-communicate the internal chamber of the barrel with the IV spike lumen and the first aperture of the IV spike, and push out the volume of the infusion liquid within the internal chamber through the IV spike lumen, out of the first aperture and into the infusion liquid container.

### BRIEF DESCRIPTION OF THE DRAWINGS

The following description of aspects of the disclosure will be better understood when read in conjunction with the appended drawings. It should be understood, however, that the disclosure is not limited to the precise arrangements and instrumentalities shown. In the drawings:

FIG. 1A is a front elevational view of an infusion bag usable with a liquid transfer device according to the present disclosure;

FIG. 1B is a front elevational view of an IV bottle usable with a liquid transfer device according to the present disclosure;

FIG. 1C is a front elevational view of a pre-filled needleless syringe and a vial usable with the liquid transfer device according to the present disclosure;

FIG. 1D is a front elevational view of an infusion set usable with the liquid transfer device according to the present disclosure;

FIG. 2 is a perspective view of a syringe plunger and an IV port of the liquid transfer device according to the present disclosure;

FIG. 3A is a perspective view of a configuration of the liquid transfer device according to the present disclosure, in a partially-locked, pre-mixing configuration;

FIG. 3B is a cross-sectional elevational view of the liquid transfer device of FIG. 3A, taken along sectional line 3B-3B of FIG. 3A;

FIG. 3C is a partial, cross-sectional elevational view of the liquid transfer device of FIG. 3A, taken along sectional line 3C-3C of FIG. 3A;

FIG. 3D is a cross-sectional elevational view of the liquid transfer device of FIG. 3A, taken along sectional line 3D-3D of FIG. 3A;

FIG. 3E is a cross-sectional, bottom plan view of the liquid transfer device of FIG. 3A, taken along sectional line 3E-3E of FIG. 3A;

FIG. 4A is a perspective view of the liquid transfer device according to the present disclosure, in a ready-to-mix configuration;

FIG. 4B is a cross-sectional elevational view of the liquid transfer device of FIG. 4A, taken along sectional line 4B-4B of FIG. 4A;

FIG. 4C is a partial, cross-sectional elevational view of the liquid transfer device of FIG. 4A, taken along sectional line 4C-4C of FIG. 4A;

FIG. 4D is a cross-sectional elevational view of the liquid transfer device of FIG. 4A, taken along sectional line 4D-4D of FIG. 4A;

FIG. 5A is a perspective view of the liquid transfer device according to the present disclosure, in a fully-locked, post-mixing configuration;

FIG. 5B is a cross-sectional elevational view of the liquid transfer device of FIG. 5A, taken along sectional line 5B-5B of FIG. 5A;

FIG. 5C is a partial, cross-sectional elevational view of the liquid transfer device of FIG. 5A, taken along sectional line 5C-5C of FIG. 5A; and

FIG. 5D is a cross-sectional elevational view of the liquid transfer device of FIG. 5A, taken along sectional line 5D-5D of FIG. 5A.

### DETAILED DESCRIPTION OF THE DISCLOSURE

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the liquid transfer device, and designated parts thereof, in accordance with the present disclosure. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

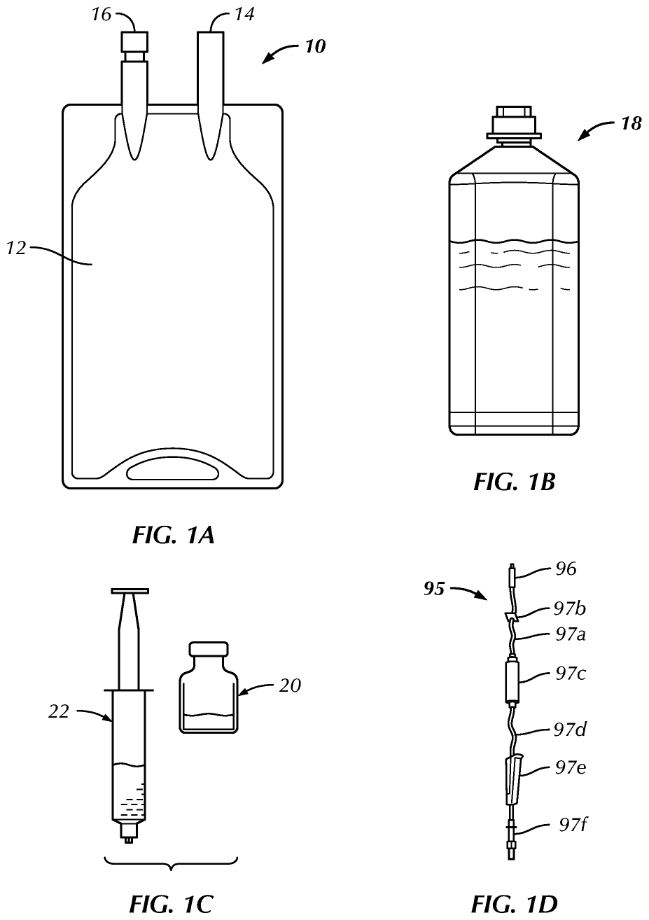

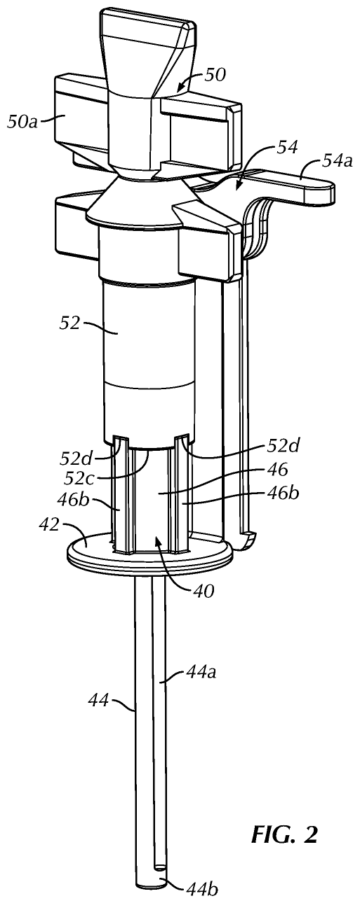

Referring to the drawings in detail, wherein like numerals indicate like elements throughout, there is shown in FIGS. 2-5D, a configuration of a liquid transfer device 30 intended for use with the combination of infusion liquid containers containing an infusion liquid and additive transfer devices. In the illustrated embodiment, the liquid transfer device 30 is intended for use with an infusion liquid container in the form of an infusion liquid bag 10 (FIG. 1A). As should be understood by those of ordinary skill in the art, a conventional infusion liquid bag 10 includes a reservoir 12 containing infusion liquid, in fluid communication with an intravenous (IV) or administration port 14 and an additive port 16. The infusion liquid bag 10 is collapsible upon administration of the infusion liquid therefrom. The liquid transfer device 30 may also, however, take a different configuration intended for use with an infusion liquid container in the form of an infusion liquid bottle 18 (FIG. 1B) or the like. The liquid transfer device 30 of the illustrated embodiment is also intended for use with an additive transfer device in the form of a sealed vial 20 (FIG. 1C). The vial 20 generally contains a medicament liquid additive or a lyophilized powder drug requiring reconstitution prior to administration to a patient, i.e., requiring mixing with the infusion liquid in the bag 10 to form a medicated infusion liquid administered to a patient. The contents of the vial 20 are, therefore, introduced into the infusion liquid bag 10 via the liquid transfer device 30 (as will be described in further detail below). The liquid transfer device 30 may also, however, take a different configuration intended for use with a syringe 22 (FIG. 1C).

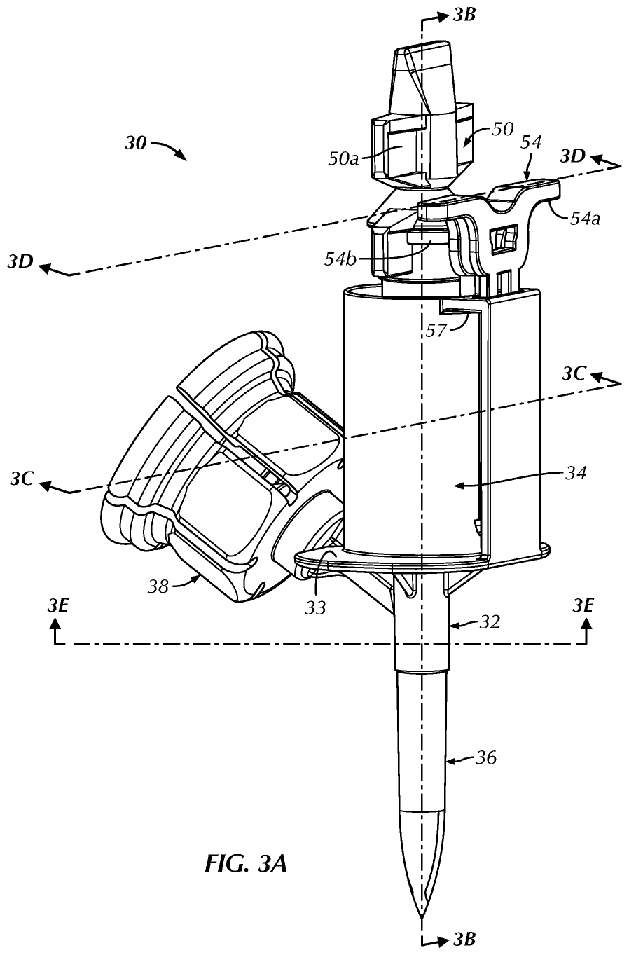

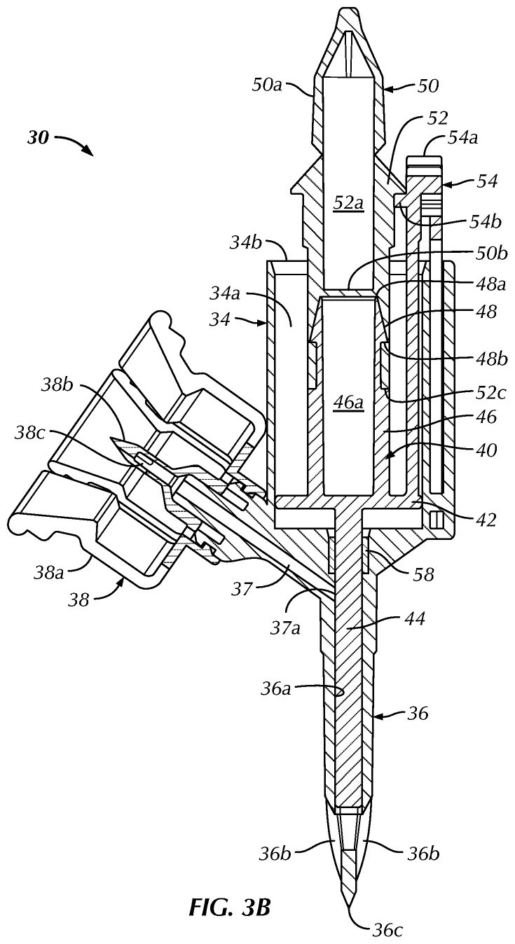

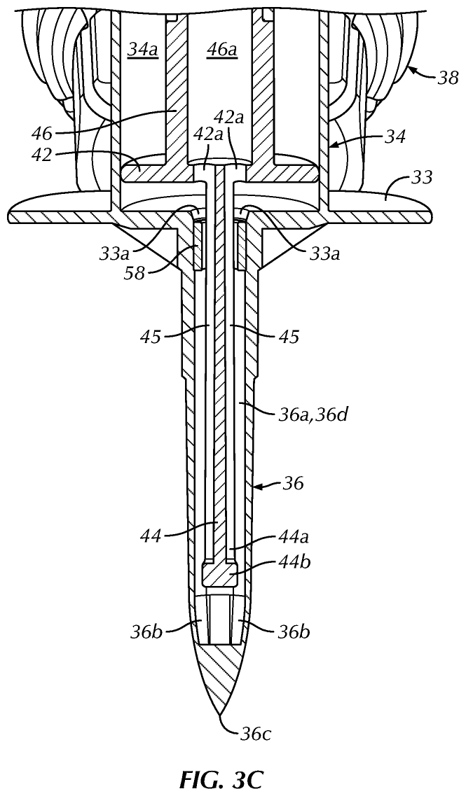

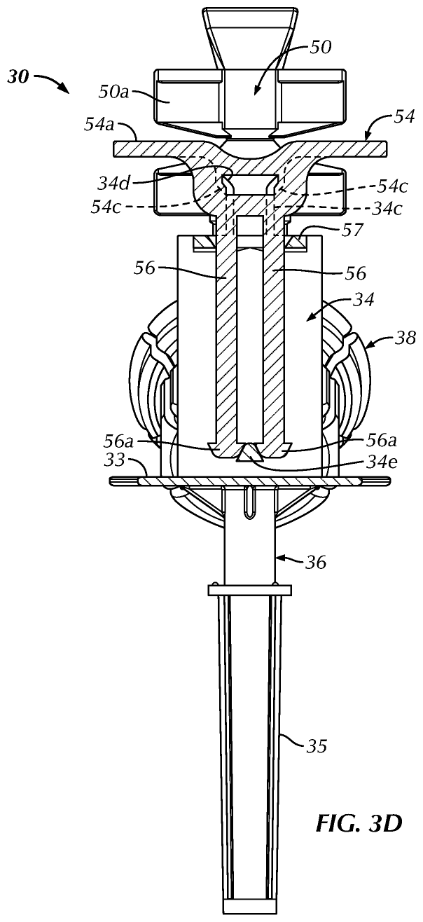

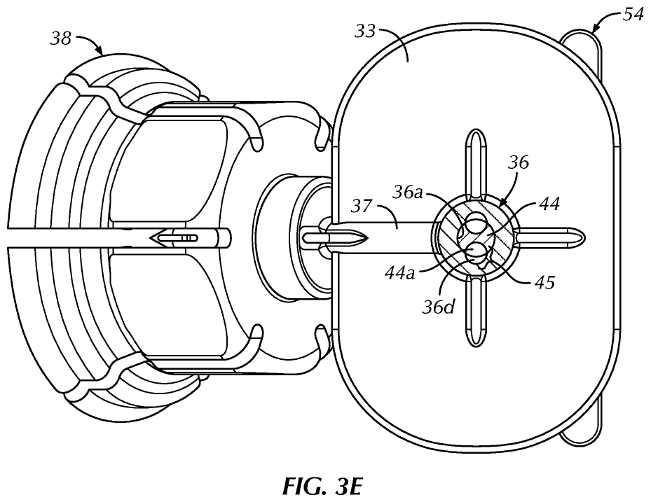

The liquid transfer device 30 includes a trifurcated connector body 32. In the illustrated configuration, the trifurcated connector body 32 includes a barrel 34 at a first end thereof, an IV spike 36 at a second end thereof, and a vial adapter 38 at a third end thereof, but the disclosure is not so limited (as described further below). The barrel 34 defines an internal chamber 34a having an open proximal end 34b for slidably receiving a plunger 40 (as will be described further below). The IV spike 36 at the second end of the trifurcated body 32 enables use of the liquid transfer device 30 with infusion liquid bags 10, i.e., for sealingly inserting the IV spike 36 into the IV port 14 of the bag 10. The IV spike 36 may be constructed from a suitable rigid metal, polymeric or plastic material, such as, for example, polycarbonate and the like. As shown best in FIGS. 3B, 3C, 4B, 4C, 5B and 5C, the IV spike 36 is co-directional with the barrel 34 and includes an internal liquid lumen 36a in fluid communication with the chamber 34a. Distal apertures 36b are peripherally disposed proximate the spike end 36c thereof. A flange 33 extends laterally from the IV spike 36 proximate an opposing end thereof, for restricting insertion depth into the IV port 14 of the bag 10. In one embodiment, the barrel 34 is integrally formed as an injection molded monolithic structure with the flange 33, but the disclosure is not so limited. A spike cap 35 (FIG. 3D) may removably cover the IV spike 36 when not in use.

A vial adapter lumen 37 angularly bifurcates from, i.e., branches off of, the IV spike lumen 36a and leads to the vial adapter 38. The vial adapter 38 is configured to mount onto a vial 20 to enable usage of the device 30 with an additive transfer device in the form of a vial 20. In the illustrated configuration, the vial adapter 38 is integrally formed at the third end of the connector body 32, but the disclosure is not so limited. The vial adapter 38 includes a flexible skirt 38a for telescopic snap fit mounting onto a vial 20 (in a standard manner) and a cannula 38b for puncturing the vial 20, e.g., via a stopper thereof, to fluidly communicate therewith. The puncturing cannula 38b includes a lumen 38c in fluid communication with the vial adapter lumen 37, and, in turn with the IV spike lumen 36a.

As shown best in FIG. 2, the plunger 40 includes a transverse piston face 42 positioned within the barrel chamber 34a (FIGS. 3B, 3C, 4B, 4C, 5B and 5C) and complementary in shape to the inner sidewall of the barrel chamber 34a, permitting sliding therein while creating a substantially air-tight seal with the sidewall. A plunger rod 44 extends distally from the piston face 42 and into the IV spike lumen 36a. An upright plunger tube 46 projects proximally from the piston face 42, having a plunger lumen 46a therein and terminating in a barbed fitting member 48 having an open end 48a.

An IV port 50 is fluidly connected with the plunger tube 46 via the barbed fitting member 48. As shown best in FIGS. 2, 3A, 3D, 4A, 4D, 5A and 5D, the IV port 50 includes a twist-off member 50a proximate a peripheral, free proximal end of the port 50, and an elongate connecting member 52 projecting distally therefrom, having an internal lumen 52a extending therethrough and terminating in an open end 52c (opposite end from the twist-off member 50a). The internal lumen 52a of the IV port 50 extends co-directionally with the plunger lumen 46a. In one embodiment, the IV port 50 may be constructed from a suitable flexible polymeric or plastic material, such as, for example, PVC, and the like. The IV port 50 includes a septum 50b positioned within the elongate connecting member 52 (see FIGS. 3B, 4B, 5B), sealing across the internal lumen 52a. Accordingly, the twist-off member 50a may be removed without leading to flow communication beyond the septum 50b. Flow communication beyond the septum 50b, i.e., with the plunger lumen 46a and beyond, is only achieved upon puncturing fully through the septum 50b (as described in further detail below). The twist off member 50a keeps the septum 50b sterile until use.

The barbed fitting member 48 is configured, i.e., size, dimension, material, relative to the internal diameter and material of the elongate connecting member 52 to advance into the internal lumen 52a through the open end 52c, and form a barbed, friction, i.e., interference, fit therebetween. As should be understood by those of ordinary skill in the art, the barbed fitting member 48 permits advancement thereof into the internal lumen 52a to sealingly and securely mount the IV port 50 co-directionally upon the plunger tube 46, and also substantially prevent withdrawal of the barbed fitting member 48 without damaging at least one of the elongate connecting member 52 and the barbed fitting member 48. That is, and as shown, the barbed fitting member 48 is frustoconically shaped, having a progressively increasing diameter in a direction away from the open end 48a. An opposing end of the barbed fitting member 48 defines a greater diameter from the underlying plunger tube 46, resulting in an annular rib 48b that bites into the interior sidewall of the elongate connecting member 52, upon attempted withdrawal of the barbed fitting member 48 out of the elongate connecting member 52. Accordingly, the barbed fitting member 48 is advanceable into the internal lumen 52a of the elongate connecting member 52 during assembly, and, thereafter, is not readily able to be withdrawn without causing damage.

As shown best in FIG. 2, the rim of the elongate connecting member 52 defining the open end 52c thereof includes at least one cutout 52d, and the plunger tube 46 includes a corresponding at least one rib 46b along an external periphery thereof and configured to mate with an opposing cutout 52d. In the illustrated embodiment, the rib 46b is elongate but may alternatively take the form of a tab or the like. In the illustrated embodiment, the elongate connecting member 52 includes a plurality of angularly spaced cutouts 52d, and the plunger tube 46 includes a corresponding plurality of angularly spaced ribs 46b. The rib(s) 46b mates with the cutout(s) 52d during mounting of the IV port 50 upon the barbed fitting member 48 to sealingly and rotationally fix the IV port 50 relative to the plunger tube 46, and, thereby to the remainder of the liquid transfer device 30. As should be understood by those of ordinary skill in the art, however, the IV port 50 may additionally or alternatively be adhesively bonded (or the like) to the plunger tube 46.

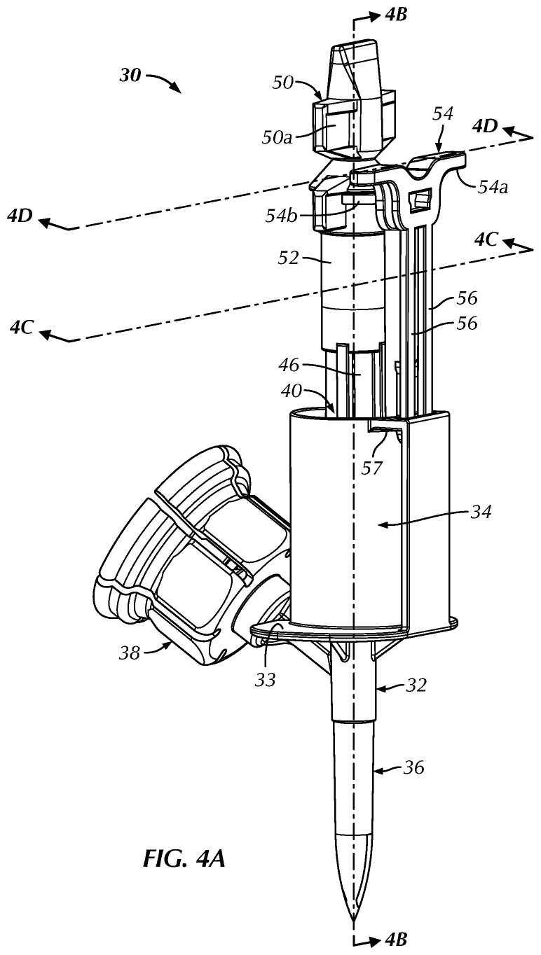

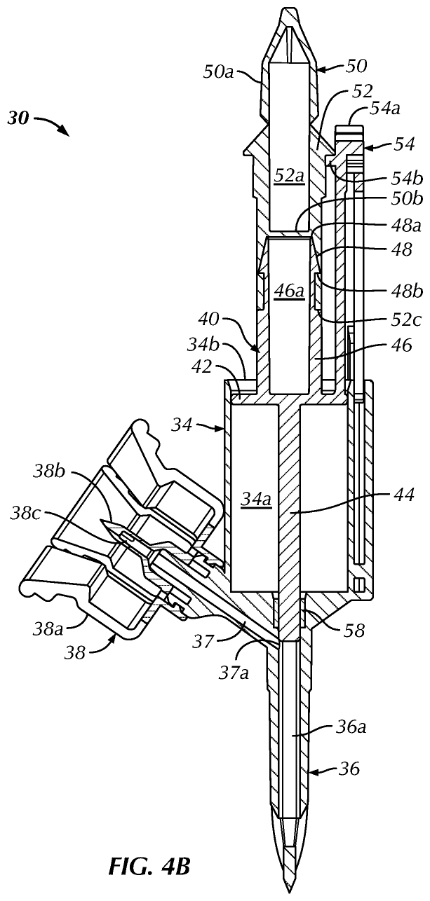

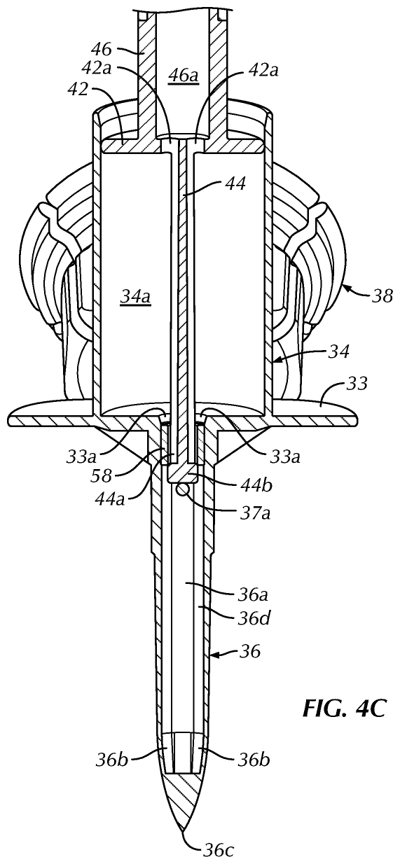

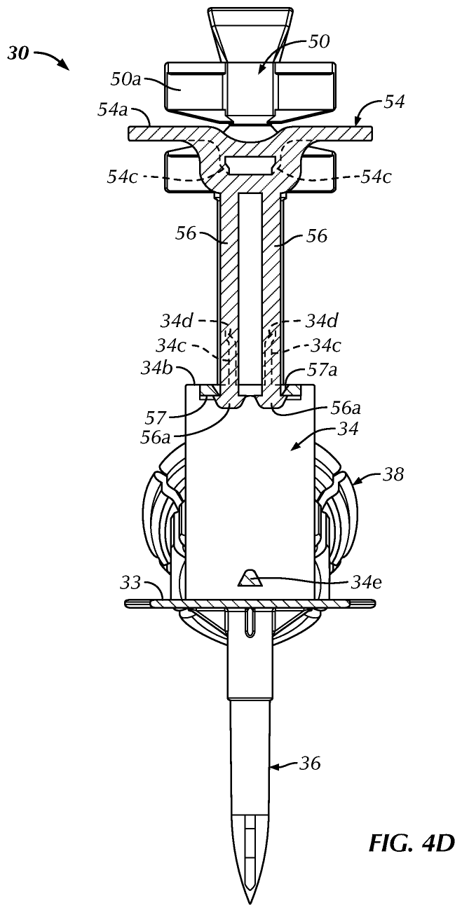

Turning to the relationship between the IV spike 36 and the plunger 40, the IV spike lumen 36a defines a generally circular cross-section, having at least one elongate, grooved channel 36d in the periphery thereof (FIG. 3E), extending from proximate the flange 33 to the distal apertures 36b (see FIGS. 3C, 4C, 5C). In the illustrated embodiment, the grooved channel is substantially semi-circular/moon-shaped in cross-section, but the disclosure is not so limited. The plunger rod 44 of the plunger 40, slidable within the IV spike lumen 36a, defines a generally complementary cross-section to the cross-section of the IV spike lumen 36a and includes a corresponding at least one elongate, grooved channel 44a in the periphery thereof, extending from the piston face 42 to a distal head 44b of the plunger rod 44 and facing/opposing the grooved channel 36d of the IV spike lumen 36a (see FIGS. 3C, 3E, 4C, 5C). The opposing grooved channels 36d and 44a define a cross-sectionally enclosed fluid-flow pathway 45 therebetween. Therefore, the plunger rod 44 substantially sealingly engages the periphery of the IV spike lumen 36a, except for the fluid-flow pathway 45. In the illustrated embodiment, the IV spike lumen 36a includes two diametrically opposed grooved channels 36d and the plunger rod 44 includes two diametrically opposed grooved channels 44a, thereby defining two diametrically opposed fluid-flow pathways 45 between the plunger rod 44 and the IV spike lumen 36a, but the disclosure is not so limited (to the number of fluid-flow pathways 45 or to being diametrically opposed). Aperture(s) 33a in the flange 33, aligned with the fluid-flow pathway(s) 45, fluidly communicate the pathway(s) 45 with the barrel chamber 34a. The distal head 44b defines a fully circular (groove-less) cross-section. As shown in FIGS. 3C, 4C, the fluid-flow pathway(s) 45 are not aligned with the vial adapter lumen 37. Thus, the plunger rod 44 may seal off the opening 37a of the vial adapter lumen 37 from fluid communication with the IV spike lumen 36a, as will be described in further detail below. As should be understood by those of ordinary skill in the art, the IV spike lumen 36a may define a different shape in cross-section and the plunger rod 44 may also define a different shape in cross-section, so long as the plunger rod 44 substantially sealingly engages the periphery of the IV spike lumen 36a, except for the fluid-flow pathway(s) 45, and the fluid-flow pathway(s) 45 are not aligned with the vial adapter lumen 37.

As shown in FIGS. 2, 3A, 3B, 3D, 4A, 4B, 4B, 5A, 5B and 5D, the liquid transfer device 30 includes a plunger handle assembly 54 fixedly attached to the plunger 40. In the illustrated embodiment, the plunger handle assembly 54 extends proximally upright from the top side of the piston face 42, having a handle 54a at a free, proximal end thereof. In one embodiment, the plunger handle assembly 54 is integrally, i.e., monolithically, formed with the piston face 42, but the disclosure is not so limited. In the illustrated embodiment, the plunger handle assembly 54 extends substantially parallel to the plunger tube 46 and the IV port 50, but the disclosure is not so limited. Optionally, the plunger handle assembly 54 includes a support 54b laterally extending from the handle 54a (or proximate the handle 54a), and into engagement with the IV port 54, e.g., snap fit onto or cradle the elongate member 52, to provide additional support for the plunger handle assembly 54.

As shown best in FIGS. 3D, 4D, and 5D, the plunger handle assembly 54 includes a pair of parallelly extending arms 56 projecting downwardly (distally) from the plunger handle 54a (outside of the barrel 34) and terminate in respective flanged ends 56a. The barrel 34 includes a pair of deflectable fingers 34c projecting upright from the open proximal end 34b thereof, and into engagement with the handle 54a. The barrel 34 further includes a stop tab 34e positioned on the exterior wall thereof, between the flanged ends 56a of the arms 56.

In use, the liquid transfer device 30 is delivered to a user in a partially-locked, pre-mixing configuration, as shown in FIGS. 3A-3D. In the pre-mixing configuration, the plunger 40 is depressed within the barrel 34 until the piston face 42 is substantially at the same elevation of the stop tab 34e. In this configuration, the plunger 40 is close to bottoming out within the barrel 34, reducing the footprint of the liquid transfer device 30 during delivery. In the partially-locked, pre-mixing configuration, the fingers 34c are positioned in an undeflected orientation and abut an underside of the handle 54a, preventing further depression (i.e., full depression) of the handle 54a and the plunger 40. The fingers 34c include laterally outwardly directly hooked terminal ends 34d, which engage corresponding downwardly and laterally inwardly directed hooks 54c projecting from an underside of the handle 54a (FIG. 3D). The engagement between the hooks 54c and the hooked terminal ends 34d stabilizes (partially-locks) the plunger 40 in the pre-mixing configuration. In the partially-locked, pre-mixing configuration, the flanged terminal ends of the arms 56 abut the stop tab 34e, positioned therebetween.

In the partially-locked, pre-mixing configuration, the liquid transfer device 30 may be coupled to an infusion liquid bag 10 via the IV spike 36 (as previously described) and with a vial 20 via the vial adapter 38 (as previously described). Thereafter, as shown in FIGS. 4A-4D, the user withdraws the plunger 40 (in a direction away from the infusion liquid bag 10) via the handle 54. Withdrawal of the plunger 40 is required to be performed with sufficient force such that the hooks 54c deflect the fingers 34c laterally inwardly (toward one another) during withdrawal and slidably disengage therefrom. The plunger 40 is fully withdrawn upon reaching a stop mechanism, which prevents inadvertent detachment of the plunger 40 from the barrel 34. In the illustrated embodiment, the stop mechanism includes a wall 57 laterally extending from the barrel 34, adjacent the open end 34b thereof (see FIGS. 3A, 4A, 5A). As shown best in FIG. 4D, the wall 57 includes an opening 57a dimensioned to permit sliding of the pair of parallelly extending arms 56 together therethrough, and to block the passage of the flanged terminal ends 56a therethrough. Therefore, the plunger 40 is stopped from further withdrawal upon contact of the flanged terminal ends 56a with the underside of the wall 57. As should be understood, however, the stop mechanism may take other forms currently known, or that later become known, capable of performing the same function of the stop mechanism described herein.

Withdrawal of the plunger 40, with the IV spike apertures 36b immersed in the infusion liquid within the bag 10, pulls fluid from the bag 10, through the apertures 36b, through the fluid-flow pathway(s) 45, i.e., between opposing grooved channels 36d and 44a, and into the barrel chamber 34a via the aperture(s) 33a. As should be understood by those of ordinary skill in the art, withdrawal of the plunger 40 creates a vacuum in the barrel chamber 34a, resulting in a pressure difference relative to the infusion liquid bag 10, thereby pulling the fluid into the barrel chamber 34a. As shown best in FIGS. 3C, 4C and 5C, a bushing 58 is positioned within the IV spike lumen 36a, adjacent the underside of the flange 33. The external periphery of the bushing 58 is configured (sized, shaped and dimensioned) to seal with the periphery of the IV spike lumen 36a. The internal aperture of the bushing 58 is configured (sized, shaped and dimensioned) to permit sliding of the plunger rod 44 therethrough and to engage the distal head 44b of the plunger rod 44, via a transition fit or a press fit, when the plunger 40 is fully withdrawn. The length of the barrel 34, the IV spike 36, the plunger rod 44 and the bushing 58, in combination with the position of the stop mechanism, are configured such that full withdrawal of the plunger 40 coincides with engagement of the distal head 44b of the plunger rod 44 with the bushing 58 (FIG. 4C), thereby sealing off the elongate, grooved channel(s) 44a of the plunger rod 44 from the elongate, grooved channel(s) 36d of the IV spike lumen 36a, and, in turn, sealing the infusion liquid within the barrel chamber 34a.

As previously described, the fluid-flow pathway(s) 45 is not aligned with the vial adapter lumen 37 and the plunger rod 44 seals off the opening 37a of the vial adapter lumen 37 from fluid communication with the IV spike lumen 36a during plunger 40 withdrawal, such that fluid may substantially only flow into the barrel chamber 34a. Upon reaching the fully withdrawn position of the plunger 40, the liquid transfer device 30 is in a ready-to-mix position. In the ready-to-mix position, as shown in FIGS. 4B and 4C, the distal head 44b of the plunger rod 44 is positional proximal to the opening 37a of the vial adapter lumen 37. That is, upon reaching the fully withdrawn position, the plunger rod 44 clears the vial adapter lumen opening 37a, thereby fluidly communicating the vial adapter lumen 37 with the IV spike lumen 36a, while sealing off the reservoir barrel 34a.

In the ready-to-mix position, the contents within the infusion liquid bag 10 are fluidly connected and mixable/combinable with the contents within the vial 20, via the fluidly connected vial adapter lumen 37 and the IV spike lumen 36a. Mixing and/or combining the contents within the infusion liquid bag 10 with the contents within the vial 20 (to create a medicated infusion liquid) is then performed. In the case that the vial 20 contains a lyophilized powder drug, the drug can be reconstituted by squeezing on the infusion liquid bag 10 to force liquid from the infusion liquid bag 10 into the vial 20.

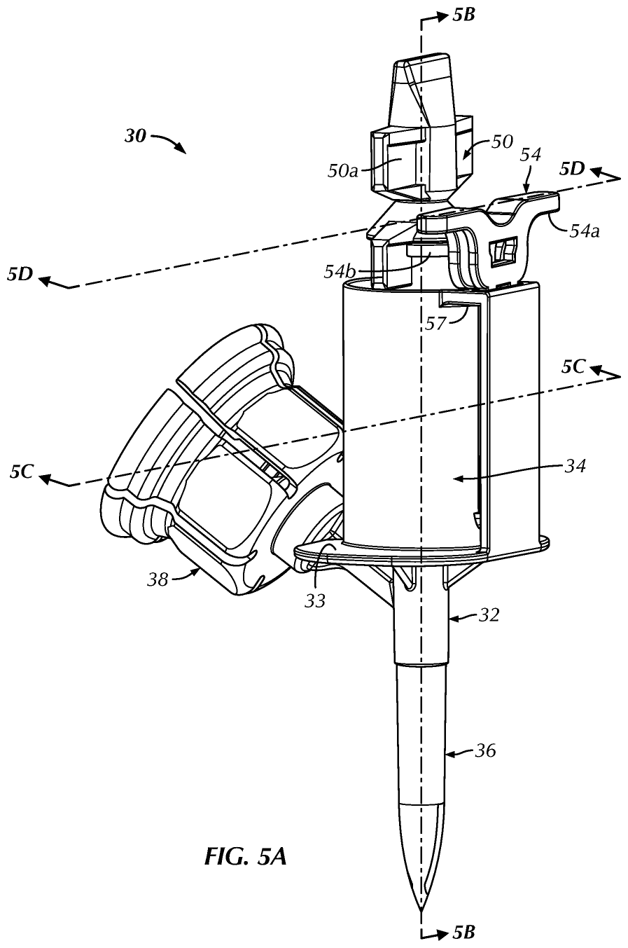

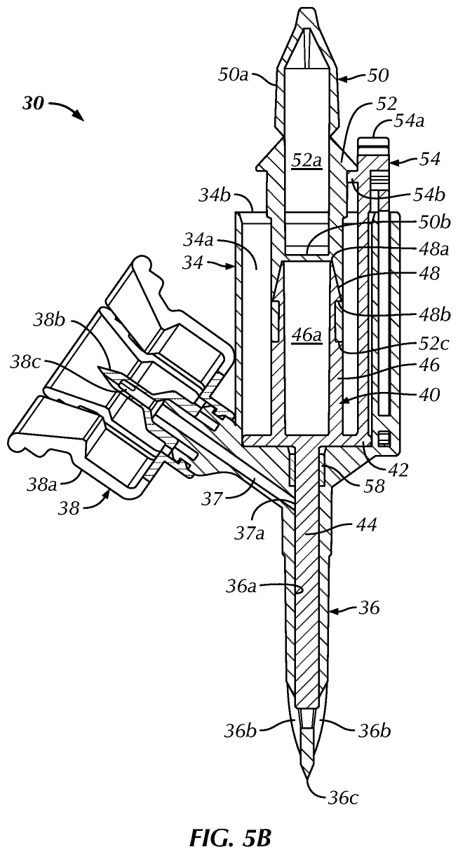

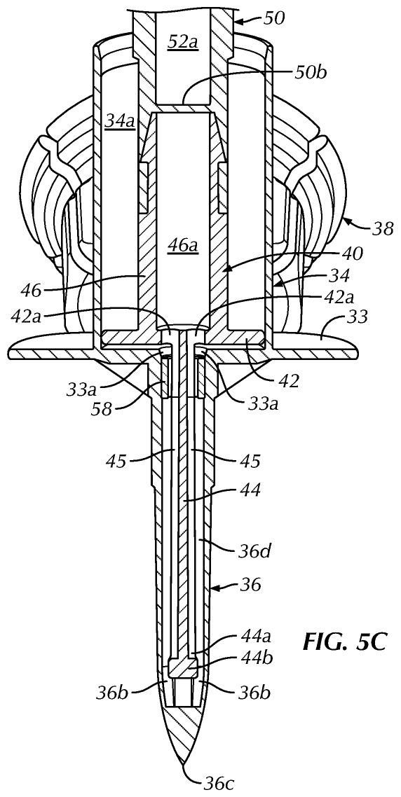

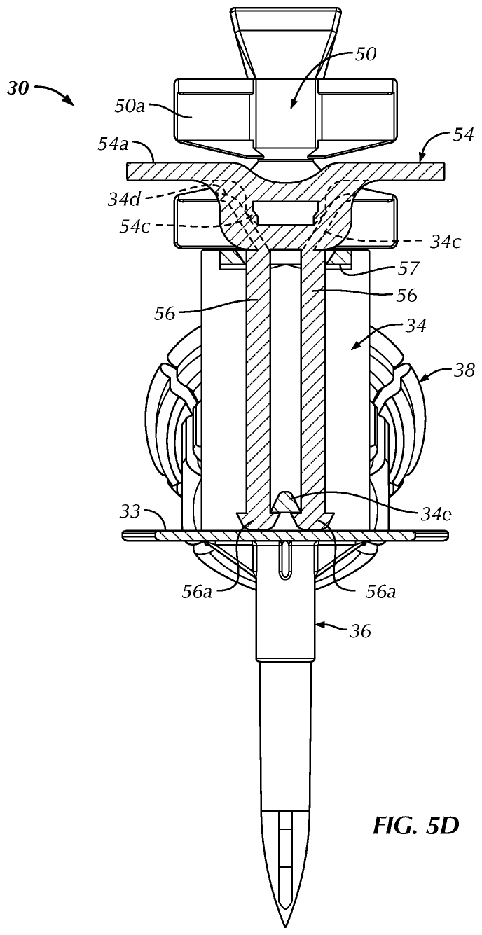

After mixing/combining the contents within the infusion liquid bag 10 and the vial 20 and orienting the liquid transfer device 30 such that the combined liquid flows into the infusion liquid bag 10, the plunger 40 is fully depressed into a fully-locked, post-mixing configuration (as shown in FIGS. 5A-5D). Depressing the plunger 40 (via the handle 54a) disengages the distal head 44b of the plunger rod 44 from the bushing 58, re-connecting the elongate, grooved channel(s) 44a of the plunger rod 44 with the elongate, grooved channel(s) 36d of the IV spike lumen 36a to fluidly communicate the barrel chamber 34a with the IV spike lumen 36a and once again sealing off the vial adapter lumen opening 37a from fluid communication with the IV spike lumen 36a. The piston face 42 pushes out the fluid within the barrel chamber 34a, through the IV spike lumen 36a and out the distal apertures 36b. Advantageously, therefore, after mixing the contents within the infusion liquid bag 10 and the vial 20, substantially any remaining unmixed medicament liquid additive or un-reconstituted lyophilized powder drug within the vial adapter lumen 37 is sealed off, and substantially any remaining unmixed medicament liquid additive or un-reconstituted lyophilized powder drug within the IV spike lumen 36a is flushed out of the lumen 36a and mixed into the infusion liquid bag 10 by the fluid within the barrel chamber 34a.

During depression of the plunger 40, the laterally inwardly directed hooks 54c projecting from an underside of the handle 54a re-engage the outwardly directly hooked terminal ends 34d of the fingers 34c and deflect the fingers 34c laterally outwardly (away from one another) (see FIG. 5D), thereby preventing the fingers 34c from abutting the underside of the handle 54a and enabling full depression of the plunger 40. When the plunger 40 is fully depressed (see FIGS. 5B, 5C), the piston face 42 bottoms out within the barrel chamber 34a, and the plunger handle assembly 54 fully locks into the post-mixing configuration via a locking mechanism. In the illustrated embodiment, the locking mechanism is activated through contact between the flanged ends 56a of the arms 56 and the intervening stop tab 34e as the plunger 40 is depressed. The flanged ends 56a and the stop tab 34e are configured such that the stop tab 34e deflects the arms 56 laterally away from one another to surpass the stop tab 34e (during plunger 40 depression) and, thereafter, the flanged ends 56a snap back around the stop tab 34e (FIG. 5D) and hook onto the underside thereof. Once the flanged ends 56a hooks onto the underside of the stop tab 34e, the plunger 40 is fully locked, i.e., the plunger 40 may not be withdrawn without causing damage to the stop tab 34e, the arms 56 or another component of the liquid transfer device 30. As should be understood by those of ordinary skill in the art, however, other locking mechanisms may be employed that are currently known, or that later become known, performing the function of the locking mechanism described herein.

As shown best in FIG. 5C, the piston face 42 includes at least one aperture 42a aligned with the aperture(s) 33a in the flange 33. Thus, in the fully-locked, post-mixing configuration of the liquid transfer device 30, the plunger lumen 46a is in fluid communication with the IV spike lumen 36a, via the aperture(s) 42a and the aperture(s) 33a (with the vial adapter lumen 37 sealed off, as previously described). In the post-mixing configuration, the twist-off member 50a is removed, providing access to the internal lumen 52a. Thereafter, an IV spike 96 of an infusion set 95 (FIG. 1D) is sealingly inserted into the internal lumen 52a and penetrates the septum 50b, thereby fluidly connecting the IV spike 96 with any remainder of the internal lumen 52a beyond the septum 50a, and, in turn, with the plunger lumen 46a and the IV spike lumen 36a for administration of the medicated infusion liquid to a patient. Conventionally, an infusion set 30 additionally includes first tubing 97a, a clamp 97b, a drip chamber 97c, second tubing 97d, a roller clamp 97e, and a male Luer connector 97f for controlling fluid administration to a patient.

It will be appreciated by those skilled in the art that changes could be made to the embodiments described above without departing from the broad inventive concepts thereof. For example, the trifurcated connector body 32 may include a vial adapter at the second end thereof (for use with an infusion liquid bottle 18) and/or a normally closed (NC) needleless additive port (not shown) at the third end thereof (enabling selection of use with a syringe 22 or with a vial 20), as described in U.S. Pat. No. 8,551,067, entitled, “Needleless Additive Control Valve,” the entire contents of which are incorporated by reference herein. As another example, the vial adapter 38 can be replaced by a manually operated stop cock, and the like. It is understood, therefore, that this invention is not limited to the particular embodiments disclosed, but it is intended to cover modifications within the spirit and scope of the present disclosure, as set forth in the appended claims.

1. A liquid transfer device configured for use with each of an infusion liquid container having an intravenous (IV) port, a vial sealed by a vial stopper, and an infusion set including an IV spike for sealing insertion into an IV port,
the liquid transfer device comprising:
a trifurcated connector body defining a barrel at a first end thereof, an IV spike at a second end thereof and a vial adapter lumen at a third end thereof,
the barrel defining an internal chamber having an open proximal end,
the IV spike defining an internal IV spike lumen having a distally located first aperture, and the IV spike being configured to sealingly insert into the IV port of the infusion liquid container, and
the vial adapter lumen branching off of the IV spike lumen;
a vial adapter secured to the vial adapter lumen and configured to mount onto the vial, the vial adapter including a cannula fluidly connected with the vial adapter lumen and configured to puncture the vial stopper upon mounting of the vial adapter onto the vial for flow communication therewith,
a plunger in slidable engagement with the internal chamber of barrel via the open proximal end thereof, the plunger comprising:
a piston face transversely oriented relative to, and in sealing engagement with, the barrel,
a tube projecting proximally from the piston face and terminating in an IV port configured to sealingly receive the IV spike of the infusion set, the tube defining an internal plunger lumen therein, and
a plunger rod projecting distally from the piston face and into the IV spike lumen, the plunger rod and the IV spike lumen defining a fluid-flow pathway therebetween, the fluid-flow pathway being sealed off from the vial adapter lumen; and
a second aperture interposed between a distal end of the internal chamber of the barrel and a proximal end of the IV spike lumen, and aligned with the fluid-flow pathway, to fluidly connect the fluid-flow pathway with the internal chamber of the barrel;
wherein:
in an advanced position of the plunger, the plunger rod seals off the vial adapter lumen from fluid communication with the IV spike lumen and the internal chamber of the barrel is in fluid communication with the IV spike lumen and the first aperture of the IV spike via the fluid-flow pathway and the second aperture, and
in a retracted position of the plunger, the plunger rod seals off the internal chamber of the barrel from fluid communication with the IV spike lumen and the first aperture of the IV spike, and the vial adapter lumen is fluidly communicated with the IV spike lumen and the first aperture of the IV spike.

2. The liquid transfer device of claim 1,
wherein the IV spike lumen includes at least one elongate grooved channel in a periphery of the IV spike lumen, extending from proximate the barrel to the first aperture, and the plunger rod includes a corresponding and opposing at least one elongate grooved channel in a periphery of the plunger rod, extending from the piston face to a distal head of the plunger rod, the at least one elongate grooved channel in the periphery of the IV spike lumen and the corresponding and opposing at least one elongate grooved channel in the periphery of the plunger rod forming the fluid-flow pathway.

3. The liquid transfer device of claim 2,
wherein the plunger rod defines a complementary cross-section to a cross-section of the IV spike lumen, other than along the at least one elongate grooved channel, such that the plunger rod substantially sealingly engages the periphery of the IV spike lumen except along the fluid-flow pathway.

4. The liquid transfer device of claim 2,
wherein the distal head of the plunger rod is positioned proximal of the vial adapter lumen in the retracted position of the plunger, thereby clearing the vial adapter lumen for fluid communication with the IV spike lumen and the first aperture of the IV spike.

5. The liquid transfer device of claim 2,
further comprising a bushing positioned within the IV spike lumen adjacent an underside of the second opening, an external periphery of the bushing being configured to seal with the periphery of the IV spike lumen and an internal aperture of the bushing being configured to permit sliding of the plunger rod therethrough and to sealing engage the distal head of the plunger rod in the retracted position thereof, thereby sealing off the internal chamber of the barrel from fluid communication with the IV spike lumen and the first aperture of the IV spike.

6. The liquid transfer device of claim 2,
wherein the distal head of the plunger rod defines a grove-less cross-section.

7. The liquid transfer device of claim 1,
wherein the piston face defines a third aperture at a distal end of the internal plunger lumen, the third aperture being aligned with the second aperture, wherein:
in the advanced position of the plunger, the internal plunger lumen is also in fluid communication with the IV spike lumen and the first aperture of the IV spike via the third aperture, the second aperture and the fluid-flow pathway, and
in the retracted position of the plunger, the plunger rod also seals off the internal plunger lumen from fluid communication with the IV spike lumen and the first aperture of the IV spike.

8. The liquid transfer device of claim 1,
wherein the fluid-flow pathway is angularly offset from the vial adapter lumen.

9. The liquid transfer device of claim 1,
wherein the IV spike is co-directional with the barrel.

10. The liquid transfer device of claim 1,
further comprising a plunger handle assembly attached to the plunger, for advancing and retracting the plunger, the plunger handle assembly being positioned outside of the barrel.

11. The liquid transfer device of claim 10,
wherein the plunger handle assembly comprises a handle and a pair of arms projecting distally from the handle, the arms terminating in respective flanged ends, and
wherein the barrel includes a stop tab positioned on an exterior wall of the barrel, aligned between the flanged ends of the arms, and a pair of deflectable fingers projecting proximally from the open proximal end of the barrel.

12. The liquid transfer device of claim 11,
wherein the plunger is distally depressed within the internal chamber of the barrel in a first configuration of the liquid transfer device, wherein the pair of deflectable fingers engage an underside of the handle and prevent further depression of the plunger and the pair of deflectable fingers are prevented from deflecting by respective distally projecting hooks of the handle.

13. The liquid transfer device of claim 12,
wherein the barrel further includes a stop mechanism extending from the exterior wall of the barrel, and wherein the plunger is proximally retractable within the internal chamber of the barrel into a second configuration of the liquid transfer device, the second configuration of the liquid transfer device corresponding generally to a maximum retraction of the plunger within the internal chamber of the barrel, wherein the flanged ends of the arms of the plunger handle assembly engage the stop mechanism.

14. The liquid transfer device of claim 13,
wherein the pair of deflectable fingers of the barrel are disengaged from the distally projecting hooks of the handle in the second configuration of the liquid transfer device, the pair of deflectable fingers being deflected away from the underside of the handle during subsequent distal depression of the plunger, thereby permitting distal depression of the plunger within the internal chamber of the barrel beyond the first configuration of the liquid transfer device and into a third configuration of the liquid transfer device, wherein the flanged ends of the arms of the plunger handle assembly surpass, and snap back onto, the stop tab, thereby locking the liquid transfer device into the third configuration, wherein the third configuration of the liquid transfer device corresponds generally to a maximum depression of the plunger within the internal chamber of the barrel.

15. A method of mixing a medicament additive contained within a vial and sealed by a vial stopper with an infusion liquid contained within an infusion liquid container, with a liquid transfer device, to form a medicated infusion liquid,
the method comprising:
sealingly inserting an IV spike of the liquid transfer device into an IV port of the infusion liquid container, the liquid transfer device comprising:
a trifurcated connector body defining a barrel at a first end thereof, the IV spike at a second end thereof and a vial adapter lumen at a third end thereof,
the barrel defining an internal chamber having an open proximal end,
the IV spike defining an internal IV spike lumen having a distally located first aperture, and
the vial adapter lumen branching off of the IV spike lumen;
a vial adapter secured to the vial adapter lumen, the vial adapter including a cannula fluidly connected with the vial adapter lumen;
a plunger in slidable engagement with the internal chamber of the barrel via the open proximal end thereof, the plunger comprising:
a piston face transversely oriented relative to, and in sealing engagement with the barrel,
a tube projecting proximally from the piston face and terminating in an IV port, the tube defining an internal plunger lumen therein, and
a plunger rod projecting distally from the piston face and into the IV spike lumen, the plunger rod and the IV spike lumen defining a fluid-flow pathway therebetween, the fluid-flow pathway being sealed off from the vial adapter lumen; and
a second aperture interposed between a distal end of the internal chamber of the barrel and a proximal end of the IV spike lumen, and aligned with the fluid-flow pathway to fluidly connect the fluid-flow pathway with the internal chamber of the barrel;
wherein:
in an advanced position of the plunger, the plunger rod seals off the vial adapter lumen from fluid communication with the IV spike lumen and the internal chamber of the barrel is in fluid communication with the IV spike lumen and the first aperture of the IV spike via the fluid-flow pathway and the second aperture, and
in a retracted position of the plunger, the plunger rod seals off the internal chamber of the barrel from fluid communication with the IV spike lumen and the first aperture of the IV spike, and the vial adapter lumen is fluidly communicated with the IV spike lumen and the first aperture of the IV spike;
mounting the vial adapter onto the vial and puncturing the vial stopper for flow communication between the vial and the vial adapter lumen;
withdrawing the plunger proximally through the internal chamber of the barrel, from the advanced position thereof toward the retracted position thereof, and, in turn, pulling a volume of the infusion liquid into the internal chamber of the barrel;
sealing off the internal chamber of the barrel from fluid communication with the IV spike lumen and the first aperture of the IV spike upon reaching the retracted position of the plunger;
combining the medicament additive contained within the vial with the infusion liquid within the infusion liquid container via fluid communication between the vial adapter lumen, the IV spike lumen and the first aperture of the IV spike when the plunger is in the retracted position, to form a medicated infusion liquid; and
depressing the plunger distally through the internal chamber to re-seal the vial adapter lumen from fluid communication with the IV spike lumen, re-communicate the internal chamber of the barrel with the IV spike lumen and the first aperture of the IV spike, and push out the volume of the infusion liquid within the internal chamber through the IV spike lumen, out of the first aperture and into the infusion liquid container.

16. The method of claim 15,
wherein the liquid transfer device further comprises a plunger handle assembly attached to the plunger and positioned outside of the barrel, and wherein the withdrawing step comprises withdrawing via the plunger handle assembly, and the depressing step comprises depressing via the plunger handle assembly.

17. The method of claim 16,
wherein the plunger handle assembly comprises a handle and a pair of arms projecting distally from the handle, the pair of arms terminating in respective flanged ends, and the barrel includes a stop tab positioned on an exterior wall of the barrel, aligned between the flanged ends of the pair of arms, a stop mechanism extending from the exterior wall of the barrel and a pair of deflectable fingers projecting proximally from the open proximal end of the barrel, wherein the withdrawing step further comprises:
withdrawing the plunger proximally through the internal chamber of the barrel until the flanged ends of the pair of arms of the plunger handle assembly engage the stop mechanism, corresponding to a maximum retraction of the plunger within the internal chamber of the barrel.

18. The method of claim 17,
further comprising the step of:
locking the plunger in place relative to the barrel, after the depressing step, to prevent subsequent movement thereof.

19. The method of claim 18,
wherein the locking step comprises depressing the plunger distally until the flanged ends of the pair of arms of the plunger handle assembly surpass, and snap back onto, the stop tab.

20. The method of claim 15,
wherein the piston face defines a third aperture at a distal end of the internal plunger lumen, the third aperture being aligned with the second aperture and fluidly connecting the internal plunger lumen with the fluid-flow pathway,
the method further comprising the step of:
sealingly inserting an IV spike of an infusion set into the IV port of the plunger after the depressing step, and, in turn, fluidly communicating the infusion set with the medicated infusion liquid within the infusion liquid container, via the first aperture of the IV spike, the fluid-flow pathway, the second aperture, the third aperture and the internal plunger lumen.
